(19) United States
(12) Patent Application Publication
(10) Pub. No.: US 2018/0228933 A1
(43) Pub. Date: Aug. 16, 2018

(54) APPARATUS AND METHOD FOR PRODUCING A PLASMA, AND USE OF SUCH AN APPARATUS

(71) Applicant: terraplasma GmbH, Garchina (DE)

(72) Inventors: Gregor Morfill, München (DE); Yangfang Li, Olching (DE); Tetsuji Shimizu, Tsukuba (JP); Bernd Steffes, Oberammergau (DE)

(21) Appl. No.: 15/750,372

(22) PCT Filed: Jul. 25, 2016
(86) PCT No.: PCT/EP2016/067698

§ 371 Date: Feb. 5, 2018

(30) Foreign Application Priority Data
Aug. 6, 2015 (DE) 10 2015 215 051.8

(51) Int. Cl. A61L 9/22; H05H 1/50; B08B 7/00; B08B 3/14; C02F 1/30; B01D 53/00

(52) U.S. Cl. CPC A61L 9/22; H05H 1/50; B08B 7/0035; C02F 2303/04; C02F 1/30; B01D 53/007; B01D 2259/818; B08B 3/14

(57) ABSTRACT

The invention relates to an apparatus (1) for producing a plasma, having at least one first electrode (3), at least one second electrode (5), which is arranged at a distance from the first electrode (3), a voltage source (7), which is connected to at least one electrode (3,59) selected from the first electrode (3) and the second electrode (5) such that a potential difference between the at least one first electrode (3) and the at least one second electrode (5) can be produced by the voltage source (7), wherein the at least one first electrode (3) and the at least one second electrode (5) define at least one discharge path (9) for an electrical discharge in a discharge region (11) between the at least one first electrode (3) and the at least one second electrode (5). In this case, a magnetic field arrangement (15) is provided that is set up and arranged relative to the at least one first electrode (3) and the at least one second electrode (5), to provide a magnetic field in the discharge region (11), so that a magnetic field vector (B) of the magnetic field is oriented at an angle to the discharge path (9).

The invention pertains to an apparatus and a method for producing a plasma as well as a use of such an apparatus.

Apparatuses and methods for producing a plasma are, in principle, well-known. In such apparatuses, typically, a discharge that is temporally and/or spatially stationary is initiated between two electrodes, and a plasma is produced by means of the discharge. Apparatuses and methods by which so-called cold, nonthermal or atmospheric plasmas are capable of being produced are particularly advantageous for the disinfection or sterilization of surfaces or fluid streams and/or the treatment of wounds. However, due to the typically provided physically stationary positioning of the discharge, for example, only comparatively small volumes of plasma can be produced or applied to fluids, or a plurality of discharges or large-area discharges are needed, which are associated with a correspondingly high application of energy and/or electrical power, and, in addition, require a complicated setup of the apparatus.

The underlying object of the invention is the creation of an apparatus and method for producing a plasma, as well as the use of such an apparatus, where the stated disadvantages do not occur.

The object of the invention is attained by creating the subjects of the independent claims. Advantageous embodiments result from the subordinate claims.

The object of the invention is attained in particular through the creation of an apparatus that is set up to produce a plasma; the apparatus provides at least one first electrode and at least one second electrode located at a distance from the first electrode. The apparatus further has a voltage source that is connected with at least one electrode, selected from the at least one first electrode and the at least one second electrode, in such a manner that a potential difference between the first and the second electrode is capable of being produced by the voltage source; the first electrode and the second electrode define at least one discharge path for an electrical discharge in a discharge region between the first electrode and the second electrode. Provision is made for a magnetic field arrangement that is set up and arranged relative to the first electrode and the second electrode in order to provide a magnetic field within the discharge region, so that a magnetic field vector of the magnetic field is oriented at an angle to the discharge path. In this manner, in the discharge region, a Lorentz force or Hall effect has an effect on the discharge; in particular, on a discharge filament developed between the electrodes, which leads to the discharge or the discharge filament becoming spatially non-stationary, and—driven by the Lorentz force—propagating within the discharge region. Through this, by means of the discharge produced between the electrodes, a larger area can be swept, compared to a volume of the discharge or the discharge filament itself, so that it is possible to cover a comparatively large volume with only one discharge, and in particular, to provide a larger plasma volume, and/or to treat a larger fluid volume. Through this, with a compact and simple as well as economical setup, the apparatus permits the provision of comparatively larger plasma volumes and/or the impinging on or treatment of comparatively larger fluid volumes.

The apparatus is preferably set up to produce a nonthermal plasma. Particularly understood under this is a plasma in which a temperature describing the distribution of the kinetic energy of the electrons of the plasma, which is also designated as the electron temperature, is not identical to and, in particular, is much greater than a temperature describing the distribution of the kinetic energy of the ions comprising the plasma, and particularly atomic or molecular ions, which is also designated as the ion temperature. Thereby, the electron temperature is much higher than the ion temperature; the ion temperature can be particularly selected to be in the range from 25° C. to at most 100° C. Such a plasma, based on the comparatively low ion temperature, can also be called a cold plasma.

Particularly preferred is the apparatus set up to produce such a nonthermal or cold plasma at atmospheric pressure, thus in particular at approximately 1013 mbar; such a plasma is also called an atmospheric-pressure plasma.

Here, particularly, plasma is designated as a material state in which charged particles exist simultaneously in the gaseous phase, with differing charges; averaged across a specific volume, a neutral electrical charge results for the observed volume. The plasma preferably comprises, in addition, non-charged atoms and/or molecules that are in electronic, vibrationally, or rotationally excited states, and which are also described as excited particles, and/or free radicals, thus altogether, especially, non-charged, reactive atoms and/or molecules, which are also designated reactive particles or reactive species.

With the term “electrode,” an electrically conductive element, onto which a potential can be applied, is particularly addressed here.

That the first electrode and the second electrode are located at a distance from each other means particularly that they are spatially separated from each other, so that, particularly, they can be connected to differing electrical potentials. The distance between the electrodes is thus particularly chosen to be large enough that they do not touch or influence each other in such a way that they can only be connected to a common, equal potential. Thereby, at the same time, the distance between the two electrodes is chosen to be at least in a range that permits the initiation of an electrical discharge between the electrodes.

In general, a voltage source is understood here to be equipment that is suitable for creating a potential difference. Thereby, it is possible that the voltage source is only connected to one of the two electrodes, namely the first electrode or the second electrode; the other of the two electrodes, namely the second electrode or the first electrode, is connected to a ground, particularly the same ground to which the voltage source is also connected. Alternatively, it is possible that the voltage source is electrically connected to both the first electrode as well as to the second electrode. It is possible that the voltage source is installed to connect one of the electrodes to a potential differing from the ground, and to ground the other of the two electrodes; it is alternatively or additionally also possible that the voltage source is installed to connect both electrodes to potentials that differ from each other, with both also differing from ground. In addition, the voltage source is preferably set up to produce a specific electrical power, so that a specific potential difference can also be maintained during current flow, namely, when a discharge between the first electrode and the second electrode occurs.

A discharge path is particularly understood here to be an intended discharge path along which a discharge between the first electrode and the second electrode is expected when a sufficiently high potential difference between the first electrode and the second electrode is produced. The discharge path is preferably the shortest connection between the first electrode and the second electrode along which the development of an electrical discharge is expected with application of a sufficiently high potential difference between the first electrode and the second electrode.

A discharge region is particularly understood to be a space between the first electrode and the second electrode, in which the at least one discharge path is located, and in which, preferably, the discharge—driven by the magnetic field—can, in particular, propagate along at least one electrode.

That a magnetic field vector is oriented at an angle to the discharge path means particularly that the magnetic field vector has a component that is perpendicular to a direction defined by the discharge path, or that the magnetic field vector is oriented completely perpendicular to the direction defined by the discharge path. In this manner, a Lorentz force has an effect on the electrically charged particles flowing along the discharge path during the electrical discharge.

The voltage source, as well as the first and the second electrode, are particularly set up so that a discharge and, therewith, particularly, a discharge filament can be produced along a discharge path in the discharge field between the first electrode and the second electrode. The first electrode, the second electrode, and the voltage source are preferably embodied as the plasma source of the apparatus.

Preferably, the first electrode and the second electrode together define a discharge plane or discharge surface for the discharge region; the discharge surface—depending on the orientation of the electrode—scan also be curved. In operation of the apparatus, the discharge filament extends across the discharge plane or discharge surface. The magnetic field vector is preferably oriented at an angle to the discharge plane or discharge surface; it is preferably perpendicular to the discharge plane or discharge surface.

According to a further development of the invention, provision is made that the voltage source is embodied as a direct current voltage source. It is particularly set up to apply to at least one of the two electrodes, namely the first electrode and/or the second electrode, a potential that is constant or essentially constant over time. The voltage source can thus be simply and economically embodied. In particular, it does not require complex, high frequency driving of the voltage source.

The voltage source is preferably set up to produce a potential difference between the first and the second electrode of at least 0.5 kV up to a maximum of 9 kV; preferably of at least 1 kV up to a maximum of 6 kV. The apparatus is preferably set up so that a flashover voltage between the first electrode and the second electrode is at most 6 kV and preferably less than 6 kV, and an operating voltage during an electrical discharge between the first electrode and the second electrode is a maximum of 4 kV and preferably less than 4 kV.

The voltage source is preferably set up to deliver a direct current of at least 0.5 mA up to a maximum of 20 mA, preferably at an operating voltage of a maximum of 4 kV, preferably of less than 4 kV, and particularly preferred, of at least 1 kV up to a maximum of 4 kV. In accordance with this, the voltage source preferably has a power output of at least 0.5 W up to a maximum of 80 W.

According to a further development of the invention, it is provided that the at least one first electrode is embodied as a rod or wire-shaped electrode, and the at least one second electrode is embodied as a ring-shaped electrode, which, seen from the direction of the circumference, encompasses the first electrode. Thereby, an axial direction is defined through an extension of the rod- or wire-shaped first electrode in the region of the second electrode, and the direction of the circumference concentrically encompasses the axial direction. Thereby, a radial direction extends perpendicular to the axial direction.

The discharge path extends in such an embodiment in a radial direction, and the magnetic field vector has at least one component in an axial direction, or extends completely in an axial direction. Correspondingly, a Lorentz force has an effect in the direction of the circumference, so that the discharge, and particularly the discharge filament, seen in the direction of the circumference and therewith along the ring-shaped second electrode, propagates in the form of an annular disc and preferably in a circular form. A propagation velocity of the discharge or the discharge filament thereby depends on the strength of the magnetic field. Effectively, and particularly in an average over time, in this manner, so to speak, a plasma sheet or a plasma surface is formed that permeates the discharge region. This phenomenon can be designated as a magnetically organized plasma sheet (MOPS).

According to a further development of the invention, it is provided that the at least one first electrode and the at least one second electrode are embodied as wire-shaped or rod-shaped electrodes. Thereby, they extend, preferably at least in a range, in a plane or along a common curved surface; the discharge region is located in this plane or surface. The magnetic field vector, then, is oriented with at least one component perpendicular to this plane or surface, so that the discharge or the discharge filament propagates along the plane or surface of the discharge region. Thus a plasma sheet or plasma surface effectively develops, particularly in an average over time, in the discharge region between the wire- or rod-shaped electrodes.

According to a further development of the invention, it is provided that the at least one second electrode is embodied as a flat electrode, which preferably has a plurality of openings. The openings can have many different geometries; for example a round, and particularly, a circular or oval geometry, or a geometry with at least a range of diverging and/or parallel walls. In this case, the first electrode is preferably embodied as a wire- or rod-shaped electrode and oriented either perpendicular to surfaces defined by the openings, and in this case a first electrode is oriented—preferably centered or central—or the first electrode is oriented in a plane defined by the openings, and extends along the openings; preferably also in this case, each opening is associated with a first electrode; however, it is also possible that a plurality of openings, and preferably all openings, are associated with a common first electrode, which, for example, can be provided with an insulating material, so that electrical discharges can only occur in the region of the openings. The first electrode is then, when it extends within a plane of an opening, preferably oriented adjacent to a first wall of the opening and opposite a second, opposite wall, and it is preferably isolated with respect to the first wall to which it is adjacent, so that a discharge is produced between the first electrode and the opposite wall of the opening.

All the various embodiments of the electrodes explained here are simply constructed and can be easily and economically manufactured. Additionally, they have specific advantages in producing a plasma, particularly with respect to a fluid stream flowing in the discharge region.

If the at least one second electrode is embodied as a flat electrode with a plurality of openings, the magnetic field vector preferably has at least one component that is oriented perpendicular to a plane or surface defined by the openings. In each case, the discharge then propagates, driven by the Lorentz force along the boundaries of the openings in a respective discharge region that is defined by the openings, and which can be preferably embodied as flat or planar.

According to a further development of the invention, it is provided that a distance between the first electrode and the second electrode along an extension of at least one of the electrodes, namely the first electrode or the second electrode, is constant. This is particularly the case when the first electrode—for example, as a rod- or wire-shaped electrode—is located in the center or midpoint of a ring-shaped second electrode and extends in an axial direction. The discharge then takes place in the sense of a circular movement within the embodied annular ring-shaped discharge region between the first electrode and the second electrode, and the discharge region has a constant radius. A constant distance between the first electrode and the second electrode can also exist when the first electrode and the second electrode are each embodied as rod- or wire-shaped electrodes; they then—always separated from each other by a constant distance—extend together along a specific path; in the simplest case, linearly. A constant distance is also possible when the second electrode is embodied as a flat electrode with a plurality of openings, namely either in the case in which the openings have a circular boundary, and the first electrode, as a wire- or rod-shaped electrode, is oriented in each case in a midpoint of such an opening and extends in an axial direction, or when the openings have at least one edge along which then—with a constant distance to the edge—the first electrode also extends. For example, in this case, the openings can have a rectangular form. A constant distance has the advantage that, in principle, the discharge can be maintained as long as a potential difference between the two electrodes, driving the discharge, is maintained by the voltage source. Thus, particularly, a continuous discharge can be run.

Alternatively or additionally, it is preferably provided that the distance between the first electrode and the second electrode along an extension diverges from at least one of the electrodes. This is particularly possible when both electrodes are embodied as wire- and/or rod-shaped electrodes; they can separate from each other along their extension, so that the distance between the electrodes increases when one of the electrodes is followed in a specific direction along its extension. However, such an embodiment can also be realized when the second electrode has a plurality of openings; at least one such opening then preferably has a correspondingly shaped edge, and, in an appropriate manner, the first electrode can be located with a diverging distance at the opening. A diverging distance between the electrodes particularly offers the possibility of allowing the discharge to end in a specific segment of the electrodes, where, namely, their distance from each other becomes too great for the discharge to still be driven by the voltage source. Thereby, the polarity of the two electrodes, in consideration of the magnetic field and the Lorentz force that produces a movement of the discharge, is preferably selected so that the discharge, which preferably develops at a location where the two electrodes have the least distance from each other, then propagates within the discharge region in the direction of the increasing distance of the electrodes from each other. Depending on the characteristic values or the set parameters of the voltage source, the discharge then reaches a specific region in which it collapses, because the voltage source can no longer drive the discharge at the distance of the electrodes from each other within this range. The discharge is then preferably newly initiated at the location at which the electrodes have their smallest distance from each other, and the process begins anew. In this manner, a quasi iterative and continuous cyclical discharge, propagating along the loading discharge region, can be produced.

Alternatively or additionally, it is preferably provided that the distance between the first electrode and the second electrode is constant within a first segment and diverges in a second segment. Thereby, the term “segment” relates here particularly to an extension of at least one of the electrodes, and particularly to the extension of both electrodes. Such an embodiment is particularly possible when both electrodes are embodied as wire- or rod-shaped electrodes, but also in a case in which the second electrode is embodied as a flat electrode with a plurality of correspondingly formed openings; the first electrode then is provided in a corresponding way in the region of the openings. The embodiment with a segment of the electrodes at a constant distance from each other, and a segment with a diverging distance from each other, has the advantage that the region where the discharge should propagate, and where it should extinguish due to the increasing distance of the electrodes from each other, can be precisely tuned.

The distance between the electrodes is preferably at least 1 mm up to at most 30 mm. Thereby, particularly, it can have a constant value in this range, or diverge within this range.

Preferably, the electrodes have, within the range of a constant distance or overall, a length of at least 1 mm, and preferably at most 1 m. Over such a range, a discharge in air with the above-stated voltage values can be maintained and propagated without difficulty Principally, however, greater distances or lengths are possible.

According to a further development of the invention, it is provided that the magnetic field arrangement has at least one permanent magnet. According to a preferred embodiment, the permanent magnet can be embodied as a ring magnet; it preferably encompasses—seen in the direction of the circumference—a ring-shaped second electrode, or it is oriented adjacent to an electrode orientation of the first electrode and the second electrode, particularly when the embodiment of the electrodes is rod- or wire-shaped, or when the second electrode is embodied as a flat electrode with a plurality of openings. In this case, it is also possible that the magnetic field arrangement has a plurality of permanent magnets that are oriented in a distribution across the flat second electrode, and particularly adjacent to the openings. In any case, it is possible that the magnetic field arrangement has a plurality of permanent magnets. Permanent magnets have the advantage that no electrical control of the magnetic field arrangement is necessary, so that the apparatus can be embodied simply and with comparatively low electrical power consumption.

Alternatively or additionally, it is possible that the magnetic field arrangement has at least one electromagnet. The advantage of an electromagnet is that the magnetic field arrangement can be turned on and off in a defined way; in addition, a simple variation of the field strength of the magnetic field arrangement is possible. Furthermore, the field strength of the magnetic field arrangement is essentially dependent on a current through the electromagnet, and is not subject to a weakening through aging effects, which can be the case with a permanent magnet.

According to a further development of the invention, it is provided that the magnetic field arrangement has at least one ring magnet. This can particularly encompass the arrangement of the at least one first electrode and the at least one second electrode—seen in the direction of the circumference—or it can be located adjacent to at least one of the electrodes, and preferably adjacent to both electrodes, that is, to that of the electrode orientation.

Alternatively or additionally, it is possible that the magnetic field arrangement has at least one electrical coil. This can also—analogously to the ring magnet—encompass in the direction of the circumference at least one of the electrodes, and preferably both electrodes, or be located adjacent to at least one of the electrodes, and preferably adjacent to both electrodes.

It is possible that the magnetic field arrangement has a plurality of ring magnets and/or a plurality of coils.

A coil is understood to be particularly an electrical induction coil, which, in its simplest form of embodiment, has a coiled electrical conductor or can comprise a coiled electrical conductor.

Alternatively or additionally, the magnetic field arrangement has a plurality of bar magnets or coils that are located along at least one electrode selected from the at least one first electrode and/or the at least one second electrode. Thereby it is possible that a plurality of bar magnets or coils are oriented around a ring-shaped second electrode and/or around at least one opening of a flat second electrode. A north pole/south pole orientation of the bar magnets and/or a longitudinal axis of the coils is thereby preferably oriented parallel to a longitudinal axis of the ring-shaped second electrode or perpendicular to the flat second electrode. It is also possible that a plurality of bar magnets and/or coils are located along a wire- or rod-shaped electrode. Thereby, in particular, the north pole/south pole orientation of the bar magnets and/or the longitudinal axis of the coils is oriented perpendicular to the extension of the electrodes.

An embodiment with a plurality of bar magnets and/or coils is possibly simpler to implement than an embodiment with one ring magnet or only one coil.

Alternatively or additionally, it is preferably provided that the magnetic field arrangement has at least one strip magnet that extends along at least one of the electrodes. This embodiment is particularly advantageous in connection with an electrode arrangement of at least two rod- or wire-shaped electrodes; such a strip magnet can be readily positioned along the extension of at least one of these electrodes. A positioning of such a strip magnet along at least one opening of a flat electrode is also easily possible.

A strip magnet is particularly understood here as a magnetic element that extends along a first direction, and the extension along the first direction is greater than a width of the element in a second direction and a height of the element in a third direction; the first direction, the second direction, and the third direction are perpendicular to each other. Such a strip magnet can, for example, be approximately 3 mm wide, between 1 cm and 2 cm high, and up to 20 cm long. Such a strip magnet represents, so to speak, an integral, sequentially positioned plurality of magnetic elements formed as one piece, and, for example, can be manufactured from a magnetic material through a sintering process.

Such a strip magnet can be particularly located along an orientation of wire- or rod-shaped electrodes, or along at least one of the edges of an opening in a flat electrode, and represents there a structurally simpler and simultaneously more advantageous embodiment, in terms of the magnetic field path, than separate bar magnets placed adjacent to each other.

According to a further development of the invention, it is provided that the apparatus has a tube segment that is in fluid contact with the discharge region, so that a fluid can be conveyed along the tube segment through the discharge region. In this manner, it is possible to treat a fluid with the plasma formed in the discharge region, and, for example, to disinfect or to sterilize, remove bad odors, remove allergens, make the fluid microbially inactive, or remove particles. Thereby, the discharge region in which the discharge propagates preferably contains a completely clear diameter of the apparatus at the location of the discharge region, so that the fluid is completely conveyed through the discharge region. In this way, it can be ensured that the entire fluid, carried through the discharge region, is impinged on by the discharge and therewith also by the plasma.

The object of the invention is also attained by creating a method for producing a plasma that has the following steps: At least one discharge filament along a discharge path in a discharge region between at least two electrodes is produced, and a magnetic field is provided that permeates the discharge region at an angle to the discharge path, and preferably perpendicular to the discharge path. Thereby, the discharge filament is driven by the magnetic field into a propagating movement within the discharge region. The method is preferably carried out by means of an apparatus according to the previously described exemplary embodiments. In particular, the advantages result that were already explained in connection with the apparatus.

A discharge filament is understood particularly to be a current of charged particles that flows along a potential gradient. Thereby, an electrical discharge between two electrodes typically occurs, in which initially a discharge channel, particularly a so-called streamer, particularly through ionization of particles along the discharge channel, is formed, before the movement of charged particles begins along the discharge channel. Charged particles then stream along the created discharge channel during the discharge, and thus form a discharge filament. The charged particles experience a Lorentz force in the magnetic field, which affects each individual particle propagating along the discharge channel, and thus, so to speak, influences the discharge filament, so that the discharge filament ultimately propagates as a whole along the discharge region.

According to a further development of the invention, it is provided that a fluid is conveyed through the discharge region. Thereby, the fluid can be impinged on in an advantageous manner with the discharge filament and particularly with the plasma that is formed by the discharge.

Preferably, the propagating movement of the discharge filament and a flow velocity of the fluid through the discharge region are tuned in such a way that every volume element of the fluid moving through the discharge region is swept at least once by the discharge filament. In this respect, it is to be determined that preferably, a periodic movement of the discharge filament is produced in the discharge region, namely, for example, according to a first embodiment of a periodic circular movement, or generally a cyclical movement, and/or according to a second embodiment, a periodic movement in such a manner that the discharge develops at a first location, then propagates along a specific path, and extinguishes at a second location—for example, through an increasing distance of the electrodes from each other. The discharge then begins anew at the first location, and the entire process repeats cyclically with a specific frequency.

A volume element of the moving fluid is understood here to be particularly a conceptual volume that moves in an axial direction—that is, perpendicular to the discharge path and particularly in the direction of the magnetic field vector—through the discharge region and has the thickness—measured in the axial direction—of the discharge filament.

In order to ensure that every volume element of the fluid moving through the discharge region is swept by the discharge at least once, this frequency of the discharge filament propagating in the discharge region is tuned in a suitable manner to the flow velocity of the fluid.

For example, for an arrangement of a ring-shaped second electrode with a rod- or wire-shaped first electrode in its center, thus particularly a discharge in the shape of an annular ring with a thickness d—measured in the axial direction—and a flow velocity of the fluid in an axial direction with a velocity v, the resulting orbital or rotational frequency of the discharge Ω must be greater than 2πv/d, in order that the discharge, from the viewpoint of the moving fluid, appears, so to speak, as a plasma sheet, and so that every volume element of the moving fluid is thus swept at least once by the discharge filament. For example, it follows, as a concrete example, for a 1 mm thickness d of the discharge measured in an axial direction, and a flow velocity of 1 m/s for the fluid, that the rotational frequency of the discharge must be greater than 6000 s⁻¹ (calculated as an angular frequency), or—in consideration of the factor 2π—must have a frequency of more than 1000 s⁻¹.

The rotational frequency of the discharge is determined by the Lorentz force and results, for example, from the following equation:

$${v}_{ϕ} = {v}_{r} ( \frac{{ω}_{e}}{{v}_{en}} ) \qquad ( 1 )$$

where v_{φ}=rΩ=2πrf is the azimuthal (circumferential) rotational velocity at a distance r from the midpoint of the cylindrical arrangement of the two electrodes 3, 5, and vᵣ is the radial expansion velocity of the discharge, which can be easily calculated from the parameters of the voltage source and preferably from the electrode distance, and further, ωₑ is the electron gyrofrequency in the magnetic field B, and wherein vₑₙ is the collision rate with atmospheric neutral particles as the dominating braking mechanism.

With the equation, it is easy to determine that rotational frequencies that must be provided for an efficient handling of a moving fluid with, for example, a flow velocity of 1 m/s, can be realized through an axial magnetic field that is greater than 0.01 T, and preferably greater than 0.1 T. However, magnetic field strengths of at least 0.1 T up to a maximum of 1 T are particularly preferred to be used. Thereby, the type of available magnets for the magnetic field arrangement still currently restricts particularly the upper limit. Naturally, greater field strengths are desirable, and will be preferentially employed to the extent that corresponding magnetic field arrangements are available. Currently, it is nevertheless quite possible, and particularly with small, preferably cylindrical neodymium permanent magnets, to provide corresponding field strengths in the range of at least 0.1 T up to a maximum of 1 T.

An embodiment of the method is preferred that is characterized in that the discharge region is held at atmospheric pressure, and preferably thus at 1013 mbar or at a locally prevailing surrounding pressure. In this way, the method is particularly inexpensive and easily implemented, because it requires neither equipment for evacuation nor equipment that would be set up to place the discharge region under elevated pressure. Thereby, with the help of the apparatus proposed here, it is easy to produce a plasma discharge at atmospheric pressure. Naturally, however, by means of the apparatus and within the scope of the method, working under other pressures, and particularly under higher or lower pressures, is also possible.

According to a further development of the invention, it is provided that a fluid, which is selected from a group consisting of air, nitrogen, a noble gas—particularly argon—carbon dioxide, a gas mixture, and a mixture of at least one gas and at least one liquid—particularly water vapor—is conveyed through the discharge region. Thereby, the terms “gas” and “liquid,” relate particularly to normal conditions; that is, they designate an aggregate state of a material, particularly at 25° C. or 298 K and 1013 mbar. The apparatus proposed here and the method proposed here can be particularly used to activate or purify fluids, particularly those selected from the previously described group.

According to a further development of the invention, it is provided that the fluid flowing through the discharge region is cleaned of a) odors, b) allergens, c) microorganisms, particularly microbes, bacteria, spores, fungi, and viruses, and/or d) particles. In this manner, the most varied fluids can be very advantageously cleaned of the most varied loads, so that the apparatus and the method are quite versatile in their application.

The object is ultimately also attained by establishing the use of an apparatus according to the previously described exemplary embodiments as an air purifying device. Thereby, the apparatus is particularly used as an air purifying device in a dwelling—for example, as a tabletop apparatus—or as part of a ventilation and air conditioning system, in a kitchen, particularly in a kitchen in a gastronomical establishment, or in a commercial kitchen, in a stable or a meat production facility, in a chemical facility, in an office, in a factory, in a sewage treatment plant, in a landfill, and/or in a medical facility such as, for example, a medical practice, a hospital ward or nursing station, or a hospital. In all of these areas, the apparatus can be utilized in a sensible, expedient, and advantageous manner, in order to clean air of pollution, particularly of the above-noted types.

A chemical facility is understood to be particularly a production facility for at least one chemical, a refinery, a production facility for at least one pharmaceutical product, a research facility, a laboratory facility, as well as an analytic laboratory facility, or another facility in which chemical experiments, tests, or reactions are carried out.

Alternatively or additionally, a use of the apparatus is recommended for the purification of a fluid from odors, allergens, microorganisms, and particularly microbes, bacteria, spores, fungi, and/or viruses, and/or particles, particularly through electronic dissociation (electron impact dissociation). Particularly for this purpose, the apparatus can be employed in an advantageous manner.

The description of the apparatus, on the one hand, as well as the method and use, on the other hand, are to be understood as complementary to each other. Characteristics of the apparatus that have been explicitly or implicitly described in connection with the method or the use are preferably characteristics that are individual or combined with each other, of a preferred exemplary embodiment of the apparatus. Procedural steps of the method or aspects of the use that have been explicitly or implicitly described in connection with the apparatus are preferably steps that are individual or combined with each other, or aspects of a preferred embodiment of the method or use. The method and/or the use preferably distinguish/es itself/themselves through at least one procedural step or one aspect that is determined by at least one characteristic of an inventive or preferred exemplary embodiment of the apparatus. The apparatus preferably distinguishes itself through at least one characteristic that is determined by at least one step or aspect of an inventive or preferred embodiment of the method or the use.

The invention is explained below in more detail with reference to the drawing. Thereby are shown in:

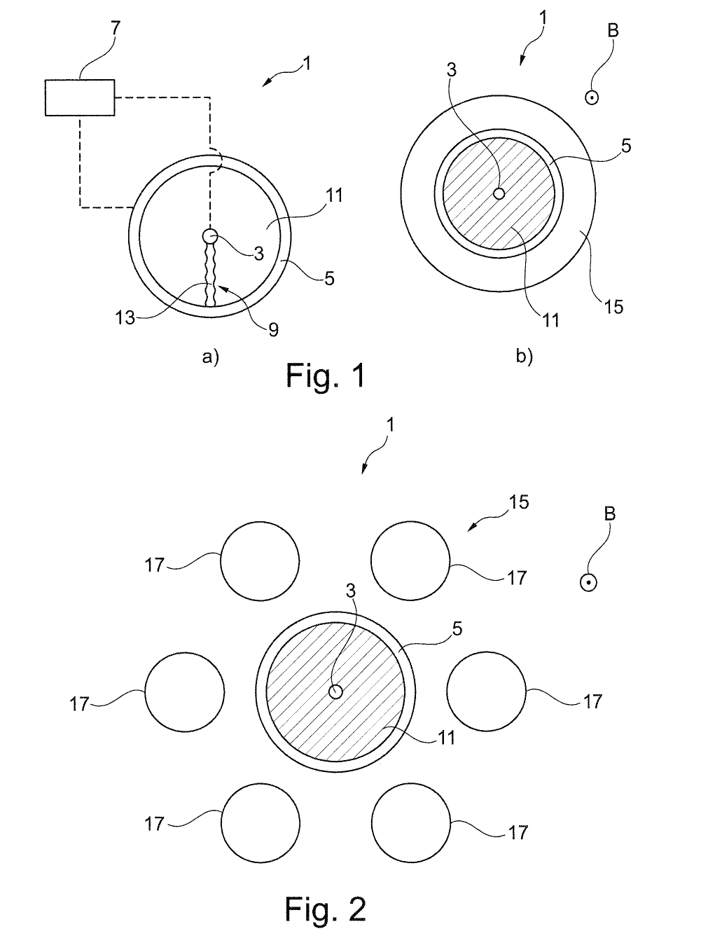

FIG. 1 A schematic illustration of a first exemplary embodiment of an apparatus;

FIG. 2 A schematic illustration of a second exemplary embodiment of the apparatus;

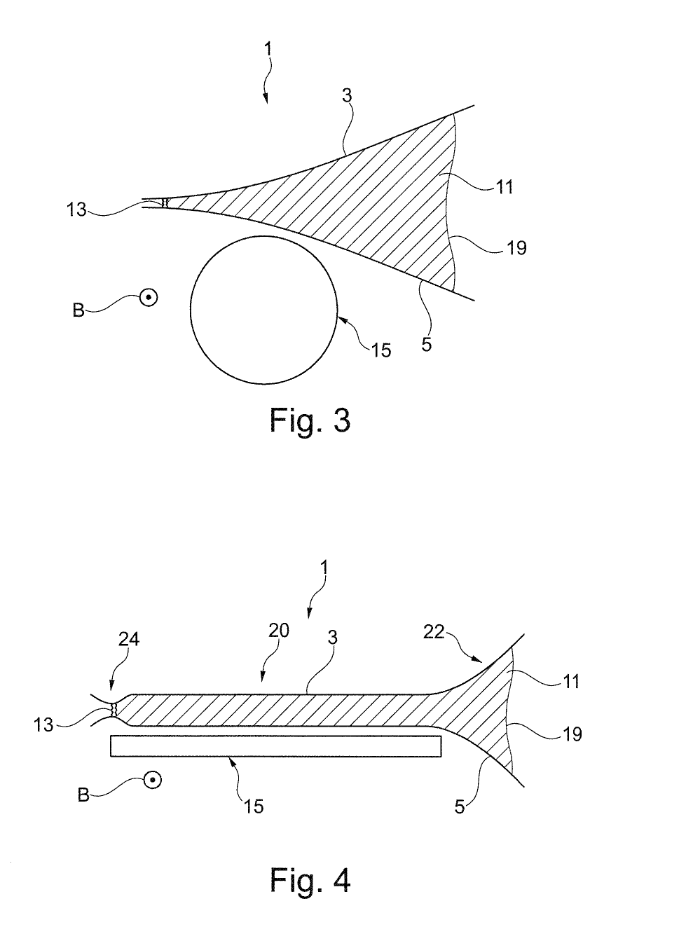

FIG. 3 A schematic illustration of a third exemplary embodiment of the apparatus;

FIG. 4 A schematic illustration of a fourth exemplary embodiment of the apparatus;

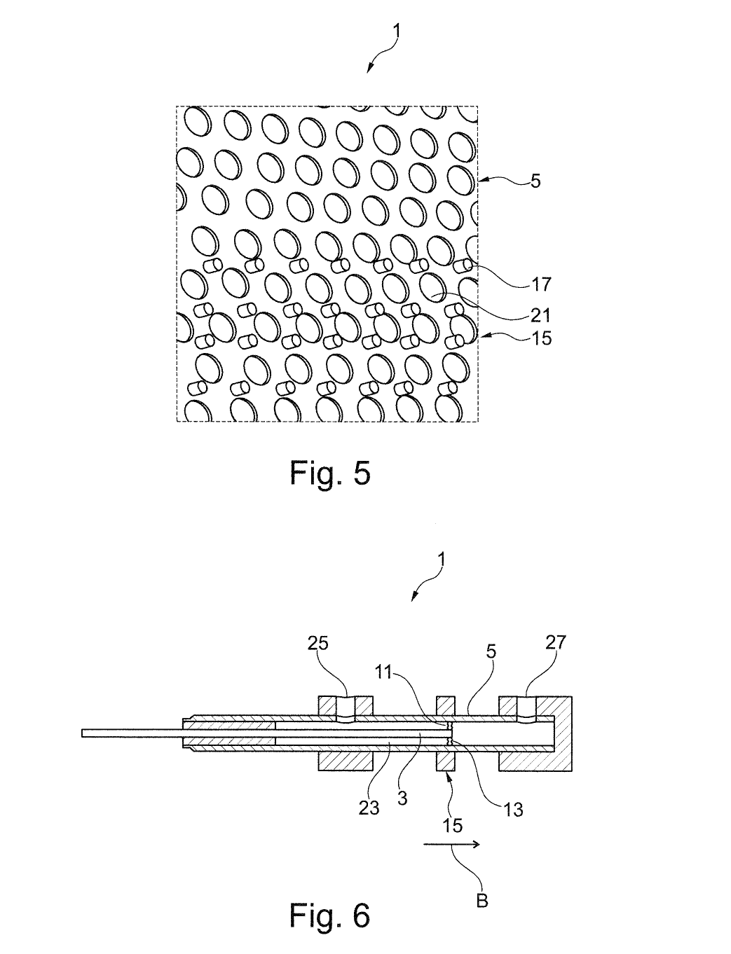

FIG. 5 A schematic illustration of a fifth exemplary embodiment of the apparatus;
and

FIG. 6 A schematic illustration of a sixth exemplary embodiment of the apparatus.

FIG. 1 shows a schematic illustration of a first exemplary embodiment of an apparatus 1 for producing a plasma. Thereby, in FIG. 1a), an apparatus 1 is illustrated that has one first electrode 3 and one second electrode 5; here, the first electrode 3 is embodied as a rod- or wire-shaped electrode, and the second electrode 5 is embodied as a ring-shaped electrode, which, seen from the direction of the circumference, encompasses the first electrode 3. Thereby, an axial direction extends perpendicular to the image plane of FIG. 1a); a radial direction is oriented perpendicular to the axial direction and located in the image plane, and a circumferential direction extends concentrically around the axial direction. The first electrode 3 and the second electrode 5 are located at a distance from each other.

In particular, the second electrode 5 is embodied here as an annular ring, and the first electrode 3 is located in the center of the circle defined by the second electrode 5. A voltage source 7 is schematically indicated as part of the apparatus 1, and which is connected with the first electrode 3 and the second electrode 5 in such a manner that a potential difference between the first electrode 3 and the second electrode 5 is capable of being produced by the voltage source 7. The first electrode 3 and the second electrode 5 define a discharge path 9 for a discharge in a discharge region 11 between the first electrode 3 and the second electrode 5. Thereby, it can be seen, on the basis of the cylindrically symmetrical setup of the arrangement of the first electrode 3 and the second electrode 5, a entire family of discharge paths 9 are defined here, which—particularly in a radial direction—extend along the circumference of the annular ring-shaped discharge region 11, and because of the constant distance between the first electrode 3 and the second electrode 5, it cannot be predicted with certainty at which point a discharge will actually initially occur, and thus which discharge path 9 from the family of possible discharge paths will be initially realized.

The voltage source 7 is preferably embodied as a direct current voltage source; it is particularly set up to produce a potential difference between the first electrode 3 and the second electrode 5 of at least 0.5 kV up to a maximum of 9 kV; preferably of at least 1 kV up to a maximum of 6 kV, and the voltage source is further preferably set up to deliver a current strength of at least 0.5 mA up to a maximum of 20 mA.

In FIG. 1a), a magnetic field arrangement is not yet illustrated, in order to explain in more detail, in steps, the mode of operation of the apparatus 1. If a discharge between the first electrode 3 and the second electrode 5 is initiated without the influence of a magnetic field—for example, with a magnetic field arrangement that has been switched off or removed—a schematically indicated discharge filament 13 develops here, that extends—except for possible interferences through air or gas movements, for example—in a radial direction within the discharge region 11. Thereby, this discharge filament 13, without the influence of a magnetic field, is stationary, in the sense that the discharge path 9, once developed, remains constant over time.

FIG. 1b) shows the apparatus 1 supplemented with a magnetic field arrangement 15 that is set up and located relative to the first electrode 3 and the second electrode 5 in such a way that it provides a magnetic field within the discharge region 11, so that a magnetic field vector B of the magnetic field is oriented at an angle, and preferably vertical, to the discharge path 9. It can be seen here that the magnetic field vector B is perpendicular to the image plane of FIG. 1. In the exemplary embodiment of the apparatus 1 concretely illustrated here, the magnetic field arrangement 15 is preferably embodied as a permanent magnet, and particularly as a ring magnet, which—seen from the direction of the circumference—encompasses the second electrode 5 and the first electrode 3. Alternatively, however, it is possible that the magnetic field arrangement 15 has a correspondingly located electromagnet, and in particular an electrical coil.

For reasons of a simplified illustration, the voltage source 7 is not shown in FIG. 1b); it is nevertheless, however, present.

If a discharge between the first electrode 3 and the second electrode 5 is now initiated, a Lorentz force has an effect on the discharge filament 13, so that the discharge filament 13 is set into rotation and propagates with a specific rotational frequency along the annular ring-shaped discharge region 11; the rotational frequency is in particular dependent on the field strength of the magnetic field. The discharge filament 13 thus sweeps across the entire discharge region 11 and forms within this—and particularly as seen in an average over time—a type of plasma sheet, and the entire discharge region 11 is impinged on by plasma.

FIG. 2 shows a schematic illustration of a second exemplary embodiment of the apparatus 1. The same and functionally same elements are provided with the same reference numerals, so that in this respect, reference is made to the previous description. The magnetic field arrangement 15 includes in this exemplary embodiment a plurality of bar magnets 17, which—preferably with the same angular distances, thus symmetrically—are located, seen from the direction of the circumference, around the first electrode 3 and the second electrode 5. The bar magnets 17 are thereby oriented so that here too, the magnetic field vector B is oriented perpendicular to the image plane of FIG. 2. Alternatively to the bar magnets 17, it is also possible that the magnetic field arrangement 15 has a plurality of electromagnets, and in particular coils, which are correspondingly located around the arrangement of the electrodes 3, 5. It is particularly preferred that the apparatus 1, and particularly the magnetic field arrangement 15, has six bar magnets 17.

It is possible that an arrangement of bar magnets 17 or an arrangement of spatially distributed coils can be more simply and more economically manufactured than a magnetic field arrangement 15 with a larger ring magnet or a correspondingly larger coil that encompasses the arrangement of electrodes 3, 5, as is illustrated in FIG. 1b).

The resulting physical effect, in consideration of the rotation of the discharge filament 13 along the discharge region 11, however, is the same in the first exemplary embodiment according to FIG. 1b) and the second exemplary embodiment according to FIG. 2. It must only be considered that typically, the magnetic field that is created by a plurality of bar magnets 17 or a plurality of coils is less homogeneous than the magnetic field created by a ring-shaped magnet or a single coil, as in the case of the first exemplary embodiment. Because of this, it is possible that in the case of the second exemplary embodiment according to FIG. 2, the discharge filament 13 exhibits varying rotational velocities along a circumferential line in the discharge region 11, because the Lorentz force also varies locally.

FIG. 3 shows a schematic illustration of a third exemplary embodiment of the apparatus 1. The same and functionally same elements are provided with the same reference numerals, so that in this respect, reference is made to the previous description. In this exemplary embodiment, the first electrode 3 and the second electrode 5 are both embodied as rod- or particularly as wire-shaped electrodes, and have a diverging distance to each other. The magnetic field arrangement 15, which preferentially here has a permanent magnet, and in particular a ring or bar magnet, is arranged here adjacent to the arrangement of the electrodes 3, 5, and namely in such a manner that here, too, the magnetic field vector B is oriented perpendicular to the image plane of FIG. 3. Alternatively, it is possible that the magnetic field arrangement 15 has an electromagnet or at least one coil. If a potential difference is applied to the electrodes 3, 5, a discharge filament 13 results, particularly at the location of the shortest distance between the electrodes 3, 5, and, by means of the magnetic field, a Lorentz force has an effect on the discharge filament 13, so that it travels along the extension of the electrodes 3, 5—here, toward the right—and sweeps the discharge region 11, until the distance between the electrodes 3, 5 becomes too great in a termination region 19, so that the voltage source 7 can no longer drive the discharge. The concrete position of the termination region 19 is particularly dependent on the voltage, and hence the potential difference, between the electrodes 3, 5. After collapse of the discharge, this starts anew at the location of the smallest distance between the electrodes 3, 5, and the previously described process repeats itself in cyclical manner. This too leads—particularly in an average over time—to an embodiment, so to speak, of a plasma sheet in the discharge region 11, that is cyclically swept by the discharge filament 13 with a specific frequency. Thereby, this frequency is particularly dependent on the field strength of the magnetic field and the length of the electrodes 3, 5 between the origination point of the discharge filament 13 and the termination region 19.

The distance between the electrodes 3, 5 is preferably between at least 1 mm and at most 30 mm in the discharge region 11.

The distance between the electrodes 3, 5 in the termination region 19 can be, for example, 3 cm.

FIG. 4 shows a schematic illustration of a fourth exemplary embodiment of the apparatus 1. The same and functionally same elements are provided with the same reference numerals, so that in this respect, reference is made to the previous description. In this exemplary embodiment as well, the electrodes 3, 5 are embodied as rod- or particularly wire-shaped electrodes; a distance between the electrodes 3, 5 in one segment is constant and in one segment is divergent; namely, it is constant in a first segment 20 and diverges in a second segment 22. Thereby, a defined location 24 is provided, at which the electrodes 3, 5 have the shortest distance from each other and where a discharge filament 13 is initially produced.

The magnetic field arrangement 15 has a strip magnet here, which extends along the arrangement of the electrodes 3, 5. Here, too, the magnetic field vector B is oriented perpendicular to the image plane of FIG. 4. The mode of operation of this exemplary embodiment of the apparatus 1 is essentially identical to the previously described mode of operation of the third exemplary embodiment according to FIG. 3; however, the result is the relatively elongated homogeneous region between the electrodes 3, 5, with a constant distance, to which the second segment with diverging electrodes 3, 5 ultimately connects, and where ultimately the termination region 19 is embodied. The first segment, in which the electrodes 3,5 have a constant distance from each other, can preferably have a length of up to 100 cm, particularly because strip magnets are currently available which, in any case, can have such a length when strung together. Thereby, the length of the region that can be realized in such a manner is particularly dependent on the length of the available strip magnets; several strip magnets can also be sequentially strung together. In this manner, there are principally no fundamental limits set for the overall length of the discharge region 11. Practical limits result, on the one hand, on the basis of sensible dimensions of the apparatus 1, and on the other hand, on the basis of desirable frequencies with which the discharge filament 13 sweeps the discharge region 11; these frequencies depend particularly on the magnetic field strength of the strip magnet and, on the other hand, on the length of the electrodes 3, 5 from the origination point 24 of the discharge filament 13 to the termination region 19. Instead of a strip magnet, it is also possible that a plurality of bar magnets are arranged next to one another, or that electromagnets, and particularly coils, are arranged in a corresponding manner next to one another, along the extension of the electrodes 3, 5.

FIG. 5 shows a schematic illustration of a fifth exemplary embodiment of the apparatus 1. The same and functionally same elements are provided with the same reference numerals, so that in this respect, reference is made to the previous description. The second electrode 5 is embodied in this exemplary embodiment as a flat electrode with a plurality of openings 21. The openings 21 are embodied here with a circular form. However, there are other openings possible, with regular or also irregular boundaries, in particular rectangular openings, or openings that have predefined irregular edge characteristics; for example, in accordance with the type of orientation of the electrodes 3, 5 in the exemplary embodiments according to FIGS. 3 and 4.

The first electrode 3 is not illustrated here. In the concretely illustrated exemplary embodiment, it is preferably provided that in each opening 21, a wire- or rod-shaped electrode is centrally oriented, and the majority of such electrodes are preferably electrically connected with each other and can be connected to a common potential that differs from the potential of the second electrode 5. In the case of other embodiments, it is also possible that the first electrode 3 extends at least in a range along at least one edge of at least one opening 21, and particularly parallel to a surface of the flat electrode 5. In this case too, it is possible that the first electrode 3 is embodied as a wire- or rod-shaped electrode.

Here, the magnetic field arrangement 15 has a plurality of bar magnets 17, of which, in order to provide better clarity, only one has been provided with the reference numeral 17. The bar magnets 17 are arranged around the openings 21, and produce preferably a magnetic field, of which the magnetic field vector is oriented vertically to a surface of the second electrode 5. The discharge filaments produced in the openings 21 then propagate in the openings 21 in a manner as was explained in connection with the FIGS. 1 through 4. In the exemplary embodiment concretely illustrated here, a behavior would particularly result as in the exemplary embodiment according to FIG. 2.

Instead of bar magnets 17, electromagnets, and particularly coils, can also be provided, or both electromagnets as well as bar magnets 17 can be provided. Alternatively, it is also possible that each opening 21, or in any case a plurality of openings 21—particularly in each case—is encompassed by a ring magnet. A common ring magnet or a common coil for a plurality of openings 21 is also possible.

Differing, adjacent magnets and/or coils can, incidentally, have magnetic fields that are oriented in parallel or antiparallel.

During operation of the apparatus 1, a fluid is preferably passed through the openings 21, and it is then treated with the plasma produced in the openings 21.

The discharge zones 11 of the other exemplary embodiments of the apparatus 1, according to FIGS. 1 through 4, preferably also have a fluid passed through them during operation, which is treated in the discharge zones 11.

If—as illustrated in FIG. 5—openings 21 with circular boundaries are selected, it is fundamentally possible to establish a 1:1 relationship from a total area of the openings 21 to a total area without openings of the second electrode 5. If openings with rectangularly shaped boundaries or openings with substantially rectangularly shaped boundaries are selected, which are then preferably combined with strip magnets, a 9:1 relationship between a total area of the openings 21 and a total area without opening of the second electrode 5 can be provided, so that, advantageously, a significantly reduced flow resistance for a fluid permeating the second electrode 5 is produced.

FIG. 6 shows a schematic illustration of a sixth exemplary embodiment of the apparatus 1. The same and functionally same elements are provided with the same reference numerals, so that in this respect, reference is made to the previous description. Apparatus 1 is embodied here as a flow tube, in which the first electrode 3 is located centrally, and preferably centered, as a rod-shaped electrode; the second electrode 5 is embodied as a ring-shaped electrode, here as a hollow, cylindrically-shaped electrode, and particularly as a cylindrical tubular element. Thereby, the second electrode 5 serves at the same time as a flow or conductive tube for a fluid, which, during operation of the apparatus 1, permeates the discharge region 11. In this respect, the apparatus 1 has a tube segment 23 that is in fluid contact with the discharge region 11, so that a fluid can be conveyed along the tube segment 23 through the discharge region 11. Thereby, the tube segment 23 is in fluid contact with a fluid inlet 25 and a fluid outlet 27, and the fluid inlet 25 and the fluid outlet 27 are particularly connecting elements to further fluid lines that are not illustrated here, such as threaded connectors for the connection of flexible tubing.

The magnetic field arrangement 15 is embodied here as a ring magnet that encompasses the arrangement of the electrodes 3, 5 in the discharge region 11.

Thereby, the mode of operation of the sixth exemplary embodiment according to FIG. 6 is identical to the mode of operation that was explained with respect to the first exemplary embodiment according to FIG. 1b), whereby a discharge propagating along the annular ring-shaped discharge region 11 is produced. The magnetic field vector B is preferably chosen parallel to the longitudinal extension of the first electrode 3, and is thus directed in an axial direction, and particularly in the longitudinal direction of the apparatus 1.

The propagating movement of the discharge filament 13 along the discharge path 11 and a flow velocity of the fluid through the discharge region 11 are preferably tuned to each other in such a way that every volume element of the fluid flowing through the discharge region 11 is swept at least once by the discharge filament 13, so that, from the viewpoint of the moving fluid, it appears, so to speak, that a discharge sheet, and particularly a plasma sheet, is embodied, through which the fluid moves and with which the fluid is treated. Thereby, the fluid is preferably cleaned of odors, of allergen, of microorganisms, and particularly of microbes, bacteria, spores, fungi, and/or of viruses, or of particles, and particularly nanoparticles, and preferably of filth or dust.

The fluid is preferably conveyed through the discharge region 11 in such a manner that, within the discharge region 11, atmospheric pressure or a pressure approximating atmospheric pressure, and preferably no coarse vacuum, and especially no medium or high vacuum, and also no high pressure, predominates.

As a fluid, preferably air, nitrogen, a noble gas such as argon, carbon dioxide, a gas mixture, or a mixture of at least one gas and at least one liquid, such as water vapor, is used.

The apparatus 1 can be used in an advantageous manner as an air purifying device, particularly for air purifying in a dwelling, in a kitchen, particularly in a commercial kitchen such as a kitchen in a gastronomic establishment, in a stable or a meat production facility, in a chemical facility, in an office, in a factory, in a sewage treatment plant, in a landfill, or in any other facility in which there is a necessity for purifying air of at least one of the previously noted materials or other materials. Under this, for example, a medical facility, such as a medical clinic or a hospital, can obviously also be included.

Altogether, it can be seen that with the apparatus 1, with the help of the method and by means of the use of apparatus 1, an effective purification of a fluid is possible with little effort and at low cost.

1. An apparatus for producing a plasma, comprising
at least one first electrode,
at least one second electrode located at a distance from the first electrode,
a voltage source, which is connected in such a manner with at least one electrode selected from the first electrode and the second electrode that a potential difference between the at least one first electrode and the at least one second electrode can be produced by the voltage source, and
the at least one first electrode and the at least one second electrode define at least one discharge path for an electrical discharge in a discharge zone between the at least one first electrode and the at least one second electrode, and
a magnetic field arrangement, that is set up and located relative to the at least one first electrode and the at least one second electrode in order to provide a magnetic field in the discharge region, so that
a magnetic field vector of the magnetic field is oriented at an angle to the discharge path.

2. The apparatus according to claim 1, wherein the voltage source is embodied as a direct current voltage source.

3. The apparatus according to claim 1, wherein:
a) the at least one first electrode is embodied as a rod or wire-shaped electrode, and the at least one second electrode is embodied as a ring-shaped electrode that—seen from the direction of the circumference—encompasses the at least one first electrode; and/or
b) the at least one first electrode and the at least one second electrode are embodied as rod- or wire-shaped electrodes, and/or
c) the at least one second electrode is embodied as a flat electrode with a plurality of openings.

4. The apparatus according to claim 1, wherein a distance between the at least one first electrode and the at least one second electrode, along the extension of at least one of the electrodes,
a) is constant, or
b) divergent, or
c) is constant in a first segment and divergent in a second segment.

5. The apparatus according to claim 1, wherein the magnetic field arrangement has at least one permanent magnet and/or at least one electromagnet.

6. The apparatus according to claim 1, wherein the magnetic field arrangement
a) has at least one ring magnet or at least one coil, and/or
b) has a plurality of bar magnets or coils, which are located along at least one electrode, selected from the first electrode and the second electrode, and/or
c) has at least one strip magnet, which is located along at least one electrode, selected from the at least one first electrode and the at least one second electrode.

7. The apparatus according to claim 1, wherein the apparatus has a tube segment that is in fluid contact with the discharge region, so that a fluid can be conveyed along the tube segment through the discharge region.

8. A method for producing a plasma, particularly by means of an apparatus according to claim 1, wherein
at least one discharge filament is produced along a discharge path in a discharge region between at least two electrodes, and
a magnetic field is provided, which permeates the discharge region at an angle to the discharge path, and
the discharge filament is driven by the magnetic field into a propagating movement within the discharge region.

9. The method according to claim 8, wherein a fluid is conveyed through the discharge region, and the propagating movement of the discharge filament and a flow velocity of the fluid through the discharge region are preferably tuned to each other in such a way that every volume element of the fluid moving through the discharge region is swept at least once by the discharge filament.

10. The method according to claim 8, wherein atmospheric pressure predominates in the discharge region.

11. The method according to claim 8, wherein air, nitrogen, a noble gas, particularly argon, carbon dioxide, a gas mixture, or a mixture of at least one gas and at least one liquid, particularly water vapor, is used as a fluid.

12. The method according to claim 8, wherein the fluid moving through the discharge region is cleaned
a) of odors,
b) of allergens
c) of microorganisms, and/or
d) of particles.

13. The use of an apparatus according to claim 1,
a) as an air purification device in
a dwelling,
a kitchen, particularly of a gastronomic establishment,
a stable or a meat production facility,
a chemical facility
an office,
a factory,
a sewage treatment plant,
a landfill, or
a medical facility, and/or
b) for the purification of a fluid of
odors,
allergens,
microorganisms, and/or
particles.
